(19) United States
(12) Patent Application Publication
(10) Pub. No.: US 2010/0089741 A1
(43) Pub. Date: Apr. 15, 2010

(54) PRODUCTION OF BIOFUELS

(76) Inventors: Marc A. Portnoff, Pittsburgh, PA (US); David A. Purta, Gibsonia, PA (US); Margaret A. Nasta, McKeesport, PA (US); Jingfeng Zhang, Gibsonia, PA (US); Faiz Pourarian, Wexford, PA (US); Richard C. Jackson, Fort Worth, TX (US)

Correspondence Address: MICHAEL BEST & FRIEDRICH LLP, 100 E WISCONSIN AVENUE, Suite 3300, MILWAUKEE, WI, 53202, US

(21) Appl. No.: 12/580,027

(22) Filed: Oct. 15, 2009

Related U.S. Application Data
(62) Division of application No. 10/274,483, filed on Oct. 17, 2002, now abandoned.

(51) Int. Cl. C07C 2/00

(52) U.S. Cl. 204/157.15

(57) ABSTRACT

A method is provided for the production of biofuels. The method includes contacting at least one of a plant oil, an animal oil and a mixture thereof with a catalyst including an acid or solid acid, thereby producing a catalyst-oil mixture. RF or microwave energy is applied to at least one of the catalyst, the plant oil, the animal oil, the mixture, and the catalyst-oil mixture to produce the biofuel. The process can be adjusted to produce gasoline, kerosene, jet fuel, or diesel range middle distillate products.

### CROSS-REFERENCE TO RELATED APPLICATIONS

This application is a divisional of U.S. patent application Ser. No. 10/274,483, filed Oct. 17, 2002, the content of which is incorporated herein by reference in its entirety.

### FIELD OF THE INVENTION

The present invention relates to an improved process for making bio-fuels, and more particularly hydrocarbons, from plant oils, animal oils and combinations thereof.

### BACKGROUND OF THE INVENTION

The use of vegetable oils for transportation fuel has been known for over 100 years with the use of peanut oil to power the first diesel engines. Vegetable oil properties are not sufficient to be a direct replacement for petroleum diesel. The vegetable oils' viscosities are too high and do not burn clean enough, leaving damaging carbon deposits on the engine. Additionally, vegetable oils gel at higher temperatures hindering their use in colder climates. These problems are minimized when the vegetable oils are blended with petroleum fuels, but still remain an impediment for long-term use in diesel engines.

Most of the prior art processes are attempts to apply petroleum processes to vegetable oils. These processes have been reported to result in low yields of hydrocarbons useful for transportation fuels. The two main problems have been the high levels of conversion of vegetable oils into gases, of little or no value, and the rapid deactivation of heterogeneous catalysts via coking mechanisms.

Another problem with vegetable oils is that their flow point temperature is higher than petroleum diesel. The relevance of this problem is that at lower temperatures approaching freezing or 0° C., vegetable oils thicken and do not flow readily. This can result in blocked fuel lines in transportation vehicles. Vegetable oils are primarily composed of triglycerides, which have long straight chain hydrocarbons attached to the glyceryl group.

Transesterification presently is the best method to convert vegetable oils into diesel compatible fuels that can be burned in conventional diesel engines. Transesterification converts vegetable oils into a biodiesel fuel. However a similar cold flow problem with conventional biodiesel fuels still remains. The relevance of this problem is that at lower temperatures, e.g. around freezing or 0° C., biodiesel also thickens and does not flow as readily. Conventional biodiesel is primarily composed of methyl esters which have long straight chain aliphatic groups attached to the carbonyl group. Also the transesterification of vegetable oils exhibits a problem of producing more than 90% diesel range fuels with little or no kerosene or gasoline range fractions.

Accordingly, an improved process for high conversions of plant, vegetable and animal oils into biofuels, and more particularly, transportation hydrocarbon fuels is desired.

### SUMMARY OF THE INVENTION

In one aspect, the invention provides a method for the production of biofuels including applying radio frequency (RF) or microwave energy (ME) to at least one of a plant oil, an animal oil and a mixture thereof to produce a biofuel.

In another aspect, the invention provides a method for the production of biofuels. The method includes contacting at least one of a plant oil, an animal oil and a mixture thereof with a catalyst including an acid or solid acid, thereby producing a catalyst-oil mixture. RF or microwave energy is applied to at least one of the catalyst, the plant oil, the animal oil, the mixture, and the catalyst-oil mixture to produce the biofuel.

In a further aspect, the invention provides an improved method of reacting a triglyceride to form carboxylic acids. The method includes contacting a triglyceride with a catalyst including an acid or solid acid and applying RF or microwave energy to at least one of the catalyst and the triglyceride to produce the carboxylic acids.

In yet another aspect, the invention provides a method of controlling a reaction between a catalyst and a feedstock. The method includes contacting the catalyst with the feedstock to form a catalyst-feedstock mixture, and applying RF or microwave energy to at least one of the catalyst, the feedstock and the catalyst-feedstock mixture. The method further includes controlling at least one of a frequency, power density, field strength, and combination thereof of the RF or microwave energy to control the reaction between the catalyst and the feedstock so as to tailor the distribution of middle distillates from gasoline to diesel.

### BRIEF DESCRIPTION OF THE DRAWINGS

FIG. 1 is a schematic diagram of a reactor configuration for the process of the present invention;

FIG. 2 is a schematic diagram of a reactor configuration for the process of the present invention with the capability of preheating the gas and liquid and of recirculating the reaction mixture or components of the reaction mixture internally and externally;

FIG. 3 is a schematic diagram of a reactor configuration for the process of the present invention having the capability of recirculating the catalyst for regeneration or recharging;

FIG. 4 is a schematic diagram for improved handling of the output for any reactor design for the process of the present invention having the capability of separating product into gas and liquid;

FIG. 5 is a schematic representation for improved handling of the output for any reactor design for the process of the present invention having the capability of gas product collection, gas product recycling, liquid product collection and liquid product recycling and a means for injecting the gas and liquid to be recycled to be injected back into the feed or input stream;

FIG. 6 is the loss tangent of soybean oil and light mineral oil as a function of frequency;

FIG. 7 is a gas chromatograph of Shellwax 750;

FIG. 8 is a gas chromatograph of catalytically cracked microwave product from Shellwax 750;

FIG. 9 is a gas chromatograph of the soybean vegetable oil feed; and

FIG. 10 is a gas chromatograph of the microwave enhanced catalytically cracked product from Test B1.

FIG. 11 is a table showing the chemical composition of soybean oil, and the catalytically cracked products.

FIG. 12 is a table showing the chemical composition of soybean oil, commercial biodiesel, and catalytically cracked products-comparing operating temperature and feed gas composition.

FIG. 13 is a table showing the chemical composition of catalytically cracked products comparing the effects of microwave power level, operating temperature and operating pressure.

### DETAILED DESCRIPTION

The present invention is directed to the efficient production of biofuels for use in transportation and heating applications. This invention employs heterogeneous catalysis and the efficient application of heat including microwave or RF energy. Microwave or RF energy is used in a novel manner, with or without a catalyst, to preferentially heat the undesirable triglyceride component of plant oil feedstocks and animal oil feedstocks to promote selective cracking.

As used herein, the term “biofuel” is meant to refer to a variety of fuels made from renewable and inexhaustible biomass resources. These biomass resources include any plant or animal derived organic matter, such as dedicated energy crops and trees, agricultural food and feed crops, agricultural crop wastes and residues, wood wastes and residues, aquatic plants, algae, plant oils, animal oils, animal tissues, animal wastes, municipal wastes, and other waste materials. Biofuels may include, but are not limited to, hydrocarbons, hydrocarbons in the middle distillate range, diesels, kerosenes, gasoline, gasoline fractions, biodiesel, biojet fuel, biogasolines and combinations thereof.

As used herein, the term “plant oil” is meant to refer to lipids derived plant sources, such as agricultural crops and forest products, as well as wastes, effluents and residues from the processing of such materials. Plant oils may include vegetable oils. Examples of plant oils may include, but are not limited to, canola oil, sunflower oil, soybean oil, rapeseed oil, mustard seed oil, palm oil, corn oil, soya oil, linseed oil, peanut oil, coconut oil, corn oil, olive oil, and combinations thereof.

As used herein, the term “lipid” is meant to refer to fatty acids from biological sources and their derivatives, most commonly esters (the reaction product of an organic acid and an alcohol) and amides (the reaction product of an organic acid and an amine). The most common class of lipid is the triglyceride, the ester product of the triple alcohol glycerin (glycerol) with fatty acids.

As used herein, the term “fatty acid” is meant to refer to organic acids synthesized in nature by both animals and plants. They typically contain a hydrocarbon group with 14 to 24 carbon atoms, although chains of 4 to 28 carbons may be found. Longer chains exist, but typically in low concentrations. The hydrocarbon group may be saturated or unsaturated.

As used herein, the term “animal oil” is meant to refer to lipids derived animal sources, as well as wastes, effluents and residues from the processing of such materials. Examples of animal oils may include, but are not limited to, animal fats, yellow grease, animal tallow, pork fats, pork oils, chicken fats, chicken oils, mutton fats, mutton oils, beef fats, beef oils, and combinations thereof.

As used herein, the term “catalyst” is meant to refer to a catalyst comprising an acid or a solid acid. Catalysts may have a catalytic site that preferentially absorbs microwaves. Catalysts may also include microwave absorbers dispersed in a mild acidity catalyst. Cracking catalysts and hydroprocessing catalysts may be employed in the methods described herein. Examples of catalysts include, but are not limited to, metal oxides, mixed metal oxides, metals, metal ions thereof, and combinations thereof More specific examples include, but are not limited to, alumina, silica, zirconium oxide, titanium oxide, zeolites, commercial ZSM-5 catalysts manufactured for example, by PQ Corporation, and combinations thereof.

A selectable distribution of biofuels (e.g. middle distillate hydrocarbons) may be produced which are useful as transportation fuels through the application of at least one of microwave energy, heat, catalysis and combinations thereof. MW or RF energy may be used in a novel method to process plant oil (including vegetable oil) feedstock, animal oil feedstock, and combinations thereof, with catalysts to selectively produce biofuels that include middle distillate hydrocarbons. Nearly complete conversion of plant oil triglycerides may be achieved. High yields of 94 wt. % or better of liquid hydrocarbons have been obtained. As an example, soy vegetable oil was converted into selectable fractions of liquid hydrocarbons including gasoline, kerosene, and diesel fractions. A high level of selectivity of liquid hydrocarbon fractions can also be controlled by process condition, for example, into more than 80 wt % of gasoline and kerosene compared to less than 20 wt % into the diesel range of hydrocarbons. Significantly less hydrocarbon gas formation is obtained compared to the results determined by F. A. Twaiq, N. A. M. Zabidi, and S. Bataia (Industrial Engineering Chemistry Research, “Catalytic Conversion of Palm Oil to Hydrocarbons: Performance of Various Catalysts,” 1999, Vol. 38, pp 3230-3237), in which microwave or RF energy was not used. Also, more selective control and production of gasoline and kerosene fractions were obtained compared to those determined by Twaiq et al. and others skilled in the art.

Without intending to be limited by the theory, novel results are believed to be due in part to the microwave and RF energy's selective cracking and isomerization of vegetable oil into lighter fractions of biofuels including biodiesel, biojet (kerosene) and biogasoline ranges useful as transportation fuels. Triglycerides are herein shown to be selective absorbers of microwave and RF energy. The application of microwave or RF energy provides a means of controlling the reaction between the catalyst and the feedstock. The proper application includes control of the microwave or RF power density or field strength, frequency, and making use of modulation techniques. Control of these parameters, in particular, using any number of modulation techniques known to those skilled in the art, such as amplitude modulation, frequency modulation, pulse width modulation and combinations thereof, is of great utility to precisely control the reaction. Nearly complete conversion of plant, vegetable and animal oil triglycerides may be achieved. High yields of 94 wt. % or better of liquid hydrocarbons are also obtained. These transportation hydrocarbon fuels have the properties of conventional petroleum hydrocarbon fuels because the vegetable oils have been significantly converted into selectable fractions of gasoline, kerosene and diesel range hydrocarbons.

Usable process conditions include temperatures of at least about 150° C., more particularly, at least about 250° C., and even more particularly, at least about 300° C. Generally, the methods are carried out at temperatures less than about 600° C., more particularly, less than about 550° C., and even more particularly, less than about 450° C. The pressure at which the methods may be practiced are generally at least a negative pressure of about 14 psig, more particularly, at least about positive 10 psig, and even more particularly, at least about positive 25 psig. Typically, the pressure is less than about positive 600 psig, more particularly, less than a positive pressure of about 450 psig, and even more particularly, less than a positive pressure of about 300 psig. RF or microwave energy at a frequency greater than or equal to about 1 MHz, and more particularly, at least about 500 MHz may generally be applied. RF or microwave energy at a frequency less than about 10,000 MHz, and more particularly less than about 3,000 MHz, of RF or microwave energy may be generally applied.

The liquid hourly space velocity (LHSV) defines the oil to catalyst ratio. LHSV is the liquid hourly space velocity defined as the ratio of the volume of oil to the volume of catalyst that passes through the catalyst on an hourly basis. The LHSV range is generally at least about 0.25 per hour, and more particularly at least about 0.50 per hour. The LHSV tends to be less than about 5.0 per hour, and more specifically, less than about 2.50 per hour.

Both an inert atmosphere of nitrogen and a reducing atmosphere of hydrogen were tested within the reaction chamber, but little difference in the product results.

Chemical components of the feedstock in conjunction with the catalyst are believed to be preferentially reacted due to absorption by both the carbonyl and carboxyl groups in feedstock and the acid sites in the catalyst, which are strong microwave absorbers compared to saturated straight chain hydrocarbons.

Plant oils and vegetable oils are primarily made up of triple esters of glycerin and fatty acids. They are comprised of triglycerides with the general formula:

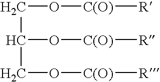

where the groups R′, R″, R′″ are straight long-chain aliphatic groups, typically containing from 8 to 22 carbon atoms. Saturated fatty acids do not contain carbon-carbon double bonds. Unsaturated fatty acids contain one or more double bonds. The catalytic reaction which produces hydrocarbons will initially break the triglycerides into carboxylic acids among other compounds. A further decarboxylation reaction is believed to occur yielding alkanes and alkenes, which are hydrocarbons, and carbon dioxide. In another mechanism to produce additional hydrocarbons, the fatty acids may condense to form anhydrides and water. The anhydrides are unstable and also convert to hydrocarbons and carbon dioxide. The glycerin segment breaks down into hydrocarbon gases.

The process for the catalytic conversion of plant oils and vegetable oils into biofuels, and more particularly, middle distillates, for the present invention can be accommodated by both batch and continuous flow reactors and systems.

Generally common to these configurations are a reaction vessel designed to permit the introduction of gas and liquid, to contain the vegetable oil feedstock and the catalyst at a suitable pressure and temperature, and that accommodates the removal of product, as shown in FIG. 1. Alternatively either gas and/or liquid may be pre-heated, depending upon process conditions, as is common practice to those skilled in the art. The catalyst is introduced into the reaction vessel and may take the form of a bed in the reaction vessel. Alternatively, the catalyst and feedstock may be circulated so that they are in close contact with each other during processing, resulting in a catalyst-feedstock (catalyst-hydrocarbon) mixture. It is known to those skilled in the art that other types of reactor catalyst beds are possible, e.g. fixed beds, moving beds, slurry reactors, fluidized beds. A gas such as nitrogen or hydrogen may be used and provision is made for recirculating the gas during the catalytic process. Such gases can be used to control and regulate system pressures. Reaction occurs on introduction of feedstock on to the catalyst within the reaction vessel. The catalyst and feedstock may be heated by heat resulting from a chemical reaction such as combustion, by resistive heating or by acoustic heating, or may be heated dielectrically by radio frequency or microwave energy. Cooling mechanisms known to those skilled in the art may be combined with the reaction vessel to accommodate exothermic reactions (e.g. the introduction of quenching gases or liquids). The reaction products may be recovered upon their removal from the vessel. The feedstock may be preheated before contact or in combination with the catalyst by heat resulting from a chemical reaction such as combustion, by resistive heating or by acoustic heating, or may be heated dielectrically by radio frequency or microwave energy.

Batch process reactors accommodating the catalyst and process of the present invention operate at elevated temperature and pressure. The batch process may have means to heat and/or cool the reactor, add and remove catalyst, receive feedstock and gas, and remove product and gas. Preferred configurations include a means to stir or recirculate the gas, catalyst and feedstock, a means to recharge the catalyst, and a means to provide RF or microwaves to the reaction site.

The preferred embodiment is a continuous flow process. Continuous flow reactors accommodating the catalyst and process of the present invention operate at elevated temperature and pressure. They may contain means to heat and/or cool the reactor, add and remove catalyst, receive feedstock and gas, preheat feedstock and gas, and remove product and gas. Preferred configurations include a means to stir or recirculate the gas, catalyst and feedstock, a means to recharge the catalyst, and a means to provide RF or microwaves to the reaction site.

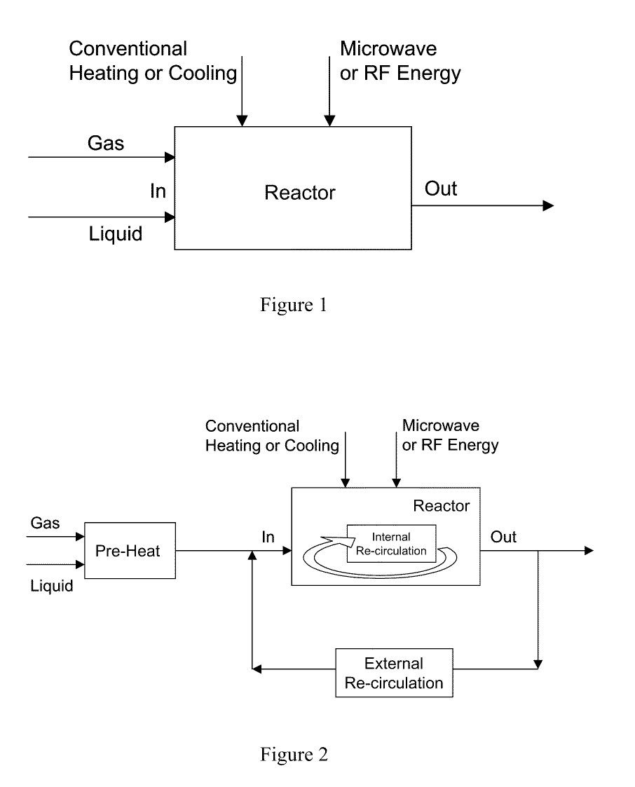

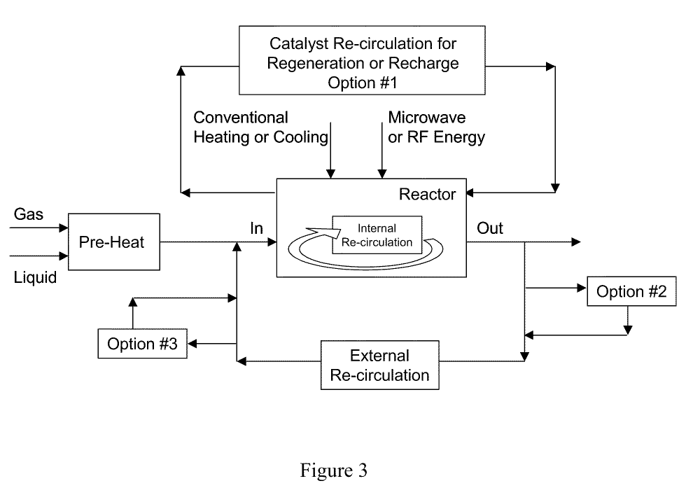

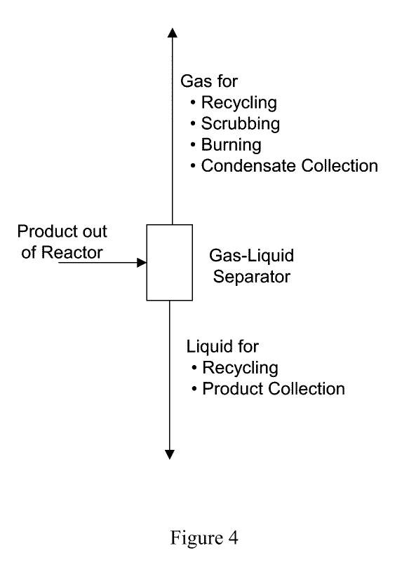

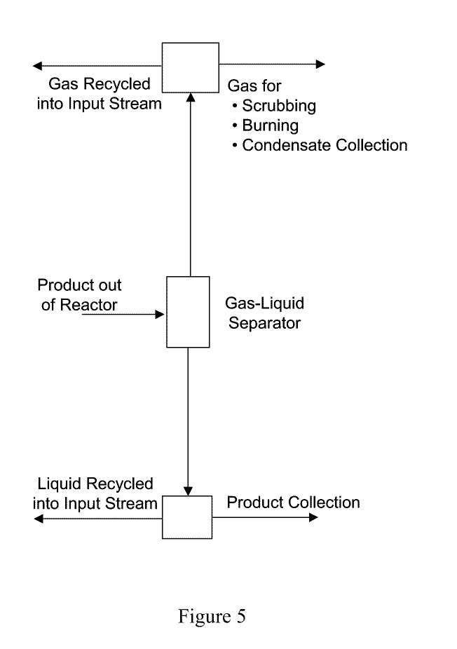

Recirculation capabilities add to the utility of reactors used in the present invention. FIG. 2 depicts the use of a reactor with the capability of preheating the gas and liquid and recirculating the reaction mixture or components of the reaction mixture internally and externally. FIG. 3 depicts the use of a reactor with the capability of recirculating the reaction mixture or components of the reaction mixture internally and externally, as well as the capability of recirculating the catalyst for regeneration or recharging. The catalyst recirculation loop for regeneration or recharge can stand alone as seen in Option 1 or be combined with existing loops as seen in Options 2 or 3. FIG. 4 depicts improved handling of the output for any reactor design of the process for the present invention having the capability of separating product into gas and liquid. The option shown in FIG. 4 can be used with any of the reactors shown in FIGS. 1, 2, and 3. FIG. 5 depicts improved handling of the output for any reactor design of the process for the present invention having the capability of gas product collection, gas product recycling, liquid product collection and liquid product recycling and a means for injecting the gas and liquid to be recycled and injected back into the feed or input stream. The option shown in FIG. 5 can be used with any of the reactors shown in FIGS. 2, 3, and 4.

### Examples

### Example 1

### Dielectric Absorption Data

Catalysis shows increased activity with increased temperature, and is generally subjected to conductively coupled conventional heating, e.g. resistive or fossil-fueled heating, to increase temperatures. Reactants and catalysts can also be heated dielectrically. Dielectric heating refers to a broad range of electromagnetic heating, either magnetically or electric field coupled, and includes radio frequency (RF) heating and microwave heating. It has been found that the value added for the process is maximized by using a minimum of dielectrically coupled energy, and by using conventional heat to supplement the total process energy. In a preferred embodiment of the present invention, microwave or RF energy is used in conjunction with fuel-fired heating or resistive heating. The exclusive use of microwave heating or RF heating, in the absence of fuel-fired heating or resistive heating, is not generally an economically viable process.

In the present process, the primary effect provided by microwave and RF energy is believed to be the enhancement of the catalyzed chemical reaction, rather than the indirect effect of heating. The dielectric parameter called the loss tangent is known by those skilled in the art to measure the relative RF or microwave energy that a particular material absorbs at a given frequency. The loss tangent, also called the loss factor, is the ratio of the energy lost to the energy stored. A larger loss tangent for a material means that more energy is absorbed relative to a material with a lower loss tangent. The dielectric absorption of energy can cause different materials to heat at substantially different rates and to achieve considerably different temperatures within the same RF or microwave field.

The dielectrically absorbed energy can also directly contribute to a process's energy balance. When used to drive an endothermic reaction, such as a cracking reaction, this means that if the absorbed RF or microwave energy equals the heat-of-reaction cracking energy, then there may not be a net increase in the bulk temperature for the process. However if more RF or microwave energy is absorbed than is necessary for the cracking reaction, then there will be a net increase in the bulk temperature.

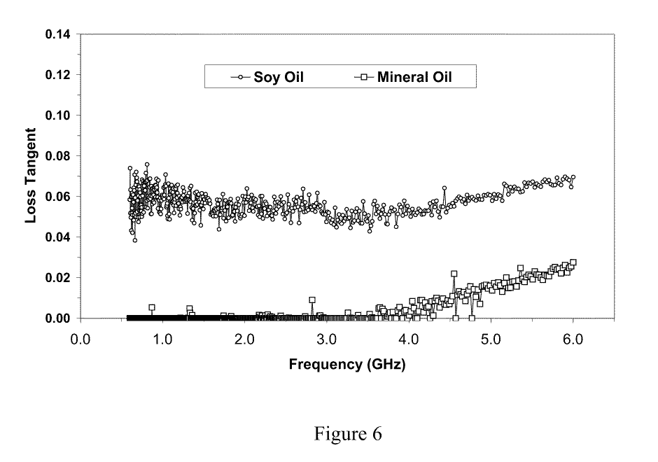

FIG. 6 provides a graph of dielectric properties of vegetable oil feedstocks, e.g. soybean oil, and a light mineral oil comprised of straight chain hydrocarbons. The dielectric loss tangent is plotted against frequency for a broad range of frequencies from 600 MHz to 6 GHz. Other plant and vegetable oils were tested and exhibited similar results including sunflower oil, peanut oil, safflower oil, corn oil, and canola oil.

The results show that the vegetable oil feedstocks selectively absorb more microwave or RF energy than the aliphatic hydrocarbons over a broad range of RF or microwave frequencies. This supports that triglycerides are the selectively stronger absorbers of microwaves or RF. Other tests show that these differences in selective absorption are relatively independent of temperature. Since the included plot shows very little dependence upon frequency, the same results for selective absorption of RF and microwave energy are also reasonably expected outside of the measured range i.e. from about 1 MHz. to beyond 10 GHz.

### Example 2

### Microwave Assisted Cracking of a Paraffin Wax

Dewaxing is the process of removing waxes from a hydrocarbon stream in order to improve low temperature properties. Waxes are high molecular weight saturated hydrocarbons or paraffins, typically those that are solid at room temperature. Dewaxing can be accomplished by solvent separation, chilling and filtering. The catalytic dewaxing process uses catalysts to selectively crack the waxes into lower molecular weight materials. This example demonstrates the use of microwaves for the application of catalytic dewaxing and cracking.

Microwave assisted cracking of C—C bonds of a high molecular weight hydrocarbon wax was demonstrated by producing a liquid from a solid hydrocarbon wax. The wax used for this demonstration was Shellwax 750. The catalyst was an ammonium Y zeolite. The solid acid catalyst along with the wax was placed into a batch process, fixed bed reactor. The ratio of wax to catalyst was at approximately one-to-one by weight. The test set up included a quartz reactor designed to operate in a 600-watt, 2.45 GHz. microwave oven, Model MDS-2000 from the CEM Corporation. The test was conducted under a slight vacuum (less than 5 psig) under a flow of argon for one to two hours. Bulk process temperatures were between 200° C. and 400° C. with temperatures rising as the wax was converted and depleted from the fixed bed reactor. Since the presence of a high temperature thermocouple can disrupt the microwave field, the temperature was measured by quickly inserting a thermocouple into the hot catalyst after opening the microwave oven door and temporarily interrupting the process. The outlet of the reactor was connected to a cold trap to condense and collect the liquid hydrocarbon products. The process commenced while the microwaves heated the wax-catalyst mixture and the evolved product was collected in the cold trap.

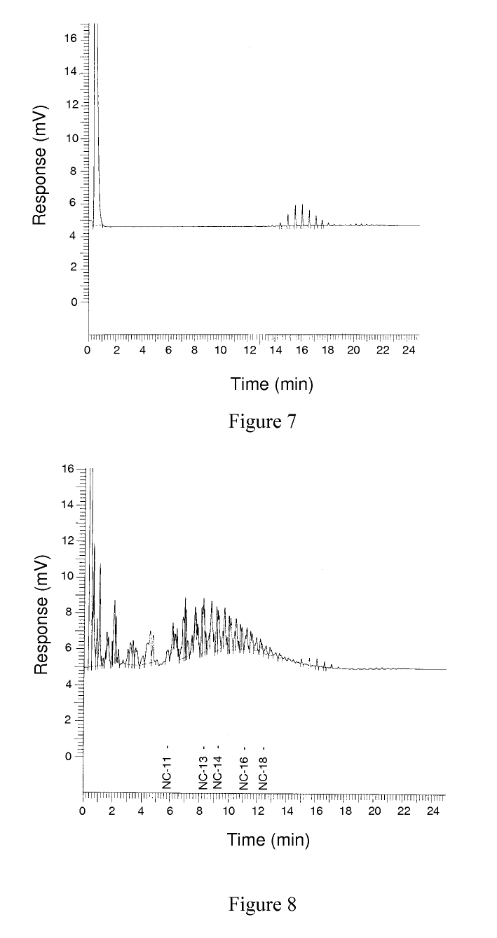

The gas chromatograph (GC) of the feed is given in FIG. 7. It shows that the original wax was composed of a hydrocarbon wax fraction in the C₂₀ to C₃₀ range. The GC trace of the resultant cracked liquid product is given in the FIG. 8. The principal hydrocarbon fraction for the product is in the C₁₀ to C₂₀ range, although there are additional lower molecular weight materials.

### Example 3

Batch Test Using Solid Catalyst with Microwaves Energy

A sequence of tests was conducted using soybean oil, as a representative vegetable oil, to demonstrate the conversion of triglycerides into middle distillate hydrocarbons.

The test apparatus included a Teflon and quartz reactor designed to operate in a 600 watt microwave oven. The reactor was instrumented with temperature and pressure sensors appropriate for operation in a microwave oven. The outlet of the reactor was connected to a cold trap to condense and collect liquid hydrocarbons. The test system allowed for periodic collection of gas samples to be analyzed via gas chromatography (GC).

Shown in this example are tests conducted under a slight vacuum (less than 12 psig) under a flow of nitrogen. Solid acid catalysts known to those skilled in the arts, such as USY and ZSM-5, along with soybean oil were placed into the reactor. The ratio of oil to catalyst was at least two to one by weight.

The microwave power density to heat the oil-catalyst mixture was estimated to range from 1-2 watts/cm³. The microwave frequency was 2.45 GHz. The pressure was approximately negative 12 psig. The oil to catalyst ratio was about 100 cc oil to about 50 cc of catalyst. The test was conducted at several different temperatures over the course of about 7 hours for Test B1 and 4 hours for Test B2. The oil-catalyst mixture was heated, using microwaves, to a set temperature and the evolved product was collected in a cold trap. The temperature was maintained for between 20 and 50 minutes to collect a sample for evaluation.

After a test, both the product's gas and liquid phases were analyzed with a GC to determine their chemical makeup and to perform a mass balance. The GC results allowed for the quantitative determination for the size range of hydrocarbons.

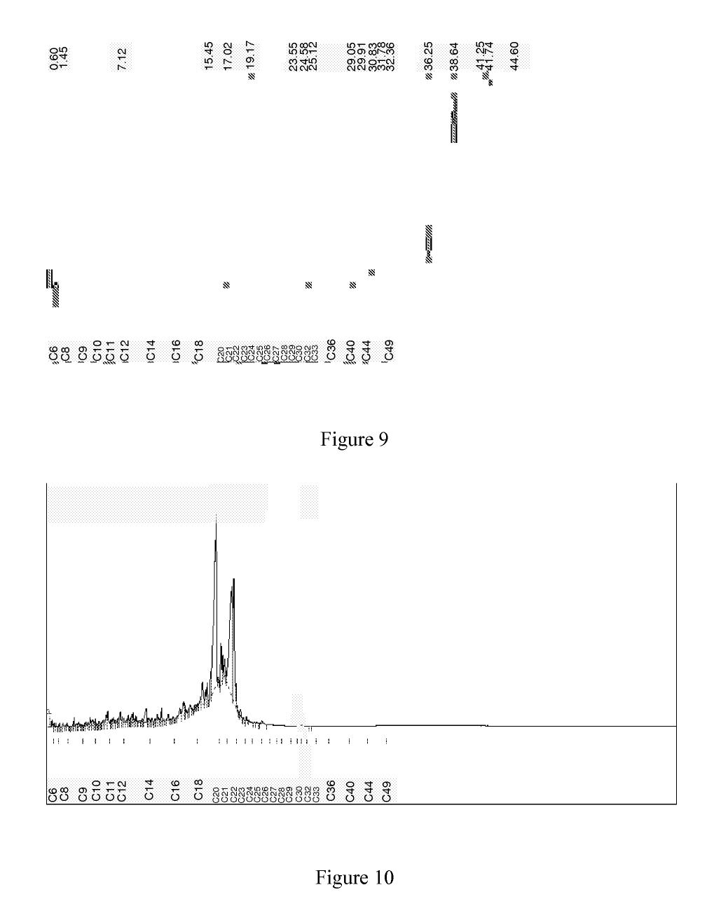

FIGS. 9 and 10 show the GC for soybean oil and product from Test B1. This product was obtained using a commercial ultra-stabilized Y (USY) zeolite extrudate, silica to alumina ratio of 12, heated using microwaves to 350° C. The plots demonstrate complete conversion of the triglycerides to middle distillate range hydrocarbons.

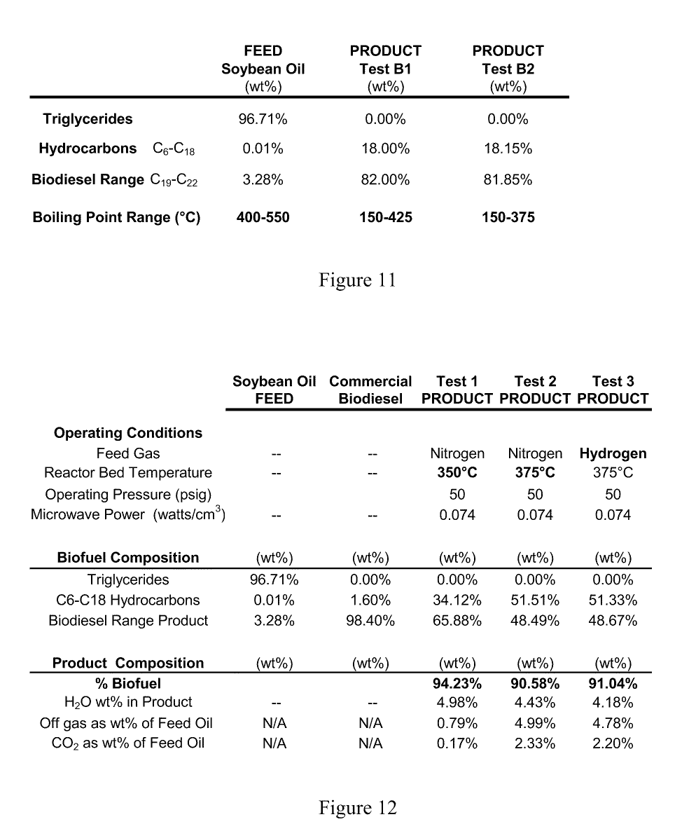

FIG. 11 shows the quantification of soybean oil, and the catalytically cracked products from the above test and a test using ZSM-5 zeolite extrudates with a silica to alumina ratio of 150. For both tests the catalyst-oil mixtures were heated to 350° C.

The significant observation from FIG. 11 is the complete conversion of triglycerides to hydrocarbons in the middle distillate range. The amount of light hydrocarbons (C₆-C₁₈) and biodiesel range hydrocarbons was approximately the same for both tests. However, the product from Test B1 had a wider boiling point range than the product from Test B2. This result is explained by the higher reactivity of the ZSM-5 catalyst over the USY catalyst.

Coking analysis was performed for the catalysts from both tests. The coke level for the USY was 8.0 wt % and for the ZSM-5 was 1.7 wt %. These coke values are well below values reported in the literature for similar test conditions.

### Example 4

### Continuous Flow Tests Using Solid Acid Catalyst Under Microwaves Energy

A series of tests were performed in a continuous flow system. Vegetable soy oil was pre-heated to a value below the reaction temperature and microwave energy was used to achieve the final reaction temperature for the catalyst and oil mixture. The microwave frequency was 2.45 GHz. For the tests reported in this example, the liquid hourly space velocity (LHSV) was fixed at a value of one. The liquid was circulated through the catalyst bed at a rate of 10 times the LHSV to simulate a stirred bed reactor. The catalyst used was a commercial ZSM-5 catalyst with a silica to alumina ratio of 50. This is a more acidic version of the ZSM-5 catalyst used in the batch test in the previous example.

To control and regulate system pressures, nitrogen was used as the feed gas for the first two tests, 1 and 2. Hydrogen was the feed gas used for the remaining tests, 3-7. For tests 1-6, the operating pressure was maintained at 50 psig. For test 7, the operating pressure was 100 psig. For all the tests, the liquid feed was pre-heated to within seven degrees of the reactor operating temperature. Three operating temperatures (e.g. 350° C., 375° C., 400° C.) were tested using either conventional heat or one of two microwave power densities of 0.074 watts/cm³ and 0.185 watts/cm³. A steady state was achieved before collecting liquid and gas samples for analysis. Mass balances were performed for all tests.

FIG. 12 summarizes the results of three tests. The table is divided into three sections: operating conditions, biofuel composition, and product composition, including gas reaction products and water. The composition of the soybean oil feed and commercial biodiesel are included for comparison. For these tests, the operating pressure and microwave power level were held constant. The process variables being evaluated include the operating temperature (e.g. 350° C., 375° C.), and the feed gas (e.g. nitrogen, hydrogen). For all three tests, 100% of the soybean oil's triglycerides were converted into lighter hydrocarbon products. The amount of C₆-C₁₈ hydrocarbons for all three tests was far greater than found in commercial biodiesel. The test results also showed that by increasing the operating temperature (Tests 1 and 2), the amount of C₆-C₁₈ hydrocarbons produced increased by over 50%. No significant difference between using nitrogen (Test 2) and hydrogen (Test 3) as the feed gas was observed.

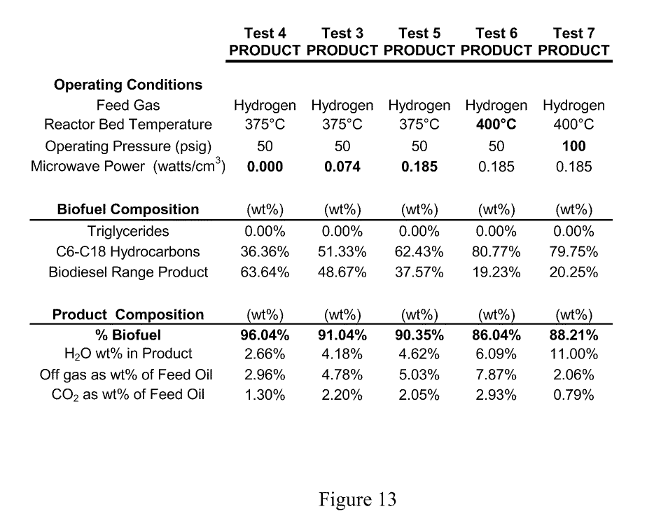

FIG. 13 summarizes the results of five tests. The table is divided into three sections: operating conditions, biofuel composition, and product composition, including gas reaction products and water. For these tests, the LHSV was set to one and the feed gas was hydrogen. The process variables evaluated include the microwave power level (0.0, 0.74, 0.185 watts/cc), operating temperature (e.g. 375° C., 400° C.), and the operating pressure (50, 100 psig). For all five tests, 100% of the soybean oil's triglycerides were converted into lighter hydrocarbon products and the amount of C₆-C₁₈ hydrocarbons for all were far greater than found in commercial biodiesel.

For tests 4, 3, 5 all processing variables were held constant except for the microwave power level. For Test 4 zero microwave power was used. For tests 3 and 5 the power level was 0.74 watts/cc and 0.185 watts/cc, respectively. The results in FIG. 13 show that the amount of C₆-C₁₈ hydrocarbons produced increase by more than 70% with increasing microwave power level. This increase in C₆-C₁₈ hydrocarbons corresponds to an increase in CO₂ and water production in agreement with reaction mechanisms for converting triglycerides to hydrocarbons.

Tests 5 and 6 compare the effect of increasing operating temperature from 375° C. to 400° C. Again, as seen previously in FIG. 12, as the operating temperature is increased, the amount of C₆-C₁₈ hydrocarbons increases. In this comparison, an increase of close to 30% is observed. Tests 6 and 7 compare the effects of increasing operating pressure from 50 psig to 100 psig. The amount of C₆-C₁₈ hydrocarbons produced remains the same as operating pressure increases. However, one can observe a slight increase in overall biofuel production, which is attributed to a threefold decrease in the off gas. The decrease in off gassing and almost doubling in the amount of water produced indicates a foreseeable change in the reaction mechanisms for producing hydrocarbons from triglycerides.

In summary, the major findings include:
The soybean oil's triglycerides were 100% converted into lighter hydrocarbon products
The 86% to 96% (weight) results for total-liquid-conversion of vegetable oils into middle distillates are higher than reported in the literature
The 1% to 8% (weight) results for off-gassing are far lower then that reported in the literature
Higher process temperatures produce lighter middle distillates
Microwave energy selectively promotes increased lighter middle distillate production at the same process temperature
No significant product differences were observed when comparing the use of hydrogen and nitrogen cover gases

These results are significant because they demonstrate that simple selection of operating parameters can efficiently control the conversion and the distribution of the middle distillates produced. This has commercial value because it enables a refinery to easily adjust the distribution of the middle distillate products over a very broad range to maximize profitability against changing market demands. Also, the lighter middle distillates from this new process can eliminate the problems associated with the cold weather properties of bio-fuel feedstocks. The cold weather properties are improved because the waxy long straight chain hydrocarbons from the plant or vegetable oils are cracked into lighter hydrocarbon products including gasoline and kerosene.

What is claimed is:

1. A method of controlling a reaction between a catalyst and a feedstock, the method comprising:
contacting the catalyst with the feedstock to form a catalyst-feedstock mixture;
applying RF or microwave energy to at least one of the catalyst, the feedstock, and the catalyst-feedstock mixture, wherein the RF or the microwave energy has a frequency, a power density, and a field strength; and
controlling at least one of the frequency, the power density, the field strength, and a combination thereof to increase the distribution of middle distillate products from the reaction.

2. The method of claim 1, wherein the frequency is controlled, and the frequency is between about 1 MHz and about 10,000 MHz.

3. The method of claim 2, wherein the frequency is between about 500 MHz and about 3,000 MHz.

4. The method of claim 1, further comprising modulating the amplitude, the frequency, or the pulse width of the RF or microwave energy.

5. The method of claim 1, wherein the power density is controlled, and the power density is between about 0.01 watts/cc and about 10 watts/cc.

6. The method of claim 1, wherein the feedstock comprises at least one of a plant oil, an animal oil and a mixture thereof.

7. The method of claim 6, wherein the plant oil comprises canola oil, sunflower oil, soybean oil, rapeseed oil, mustard seed oil, palm oil, corn oil, soya oil, linseed oil, peanut oil, coconut oil, olive oil, or combinations thereof.

8. The method of claim 6, wherein the animal oil comprises at least one of animal fat, yellow grease, animal tallow, pork fat, pork oil, chicken fat, chicken oil, mutton fat, mutton oil, beef fat, beef oil, or combinations thereof.

9. The method of claim 1, wherein the method exhibits increased production of hydrocarbons in C₆ through C₁₈ range as compared to methods in which the RF or microwave energy is not applied.

10. The method of claim 1, wherein the catalyst exhibits reduced coking as compared to methods in which the RF or microwave energy is not applied

11. The method of claim 1, wherein the catalyst comprises at least one metal oxide.

12. The method of claim 10, wherein the catalyst comprises at least one of alumina, silica, zirconium oxide, magnesium oxide, titanium oxide, and mixtures thereof.

13. The method of claim 1, wherein the catalyst comprises a zeolite.

14. The method of claim 1, wherein the middle distillate products comprise at least one of diesel, kerosene, and gasoline fractions.

15. The method of claim 1, wherein the method is performed at an operating pressure, and the operating pressure is adjusted between a negative pressure of about 14 psig and a positive pressure of about 600 psig.

16. The method of claim 15, wherein the method is performed at an operating pressure, and the operating pressure is adjusted between a positive pressure of about 25 psig and a positive pressure of about 300 psig.

17. The method of claim 1, wherein the method is performed at an operating temperature between about 150° C. and about 600° C.

18. The method of claim 17, wherein the operating temperature is between about 300° C. and about 450° C.

19. The method of claim 1, wherein the method has a liquid hourly space velocity (LHSV) corresponding to the rate at which the feedstock contacts the catalyst, and the LHSV is between about 0.25 to about 5.00 per hour.

20. A method for the production of hydrocarbons as biofuels, the method comprising:
A) heating at least one of a plant oil, an animal oil and a combination thereof to a temperature of at least about 250 ° C. with conventional heating;
B) contacting at least one of the plant oil, the animal oil and the combination thereof with a catalyst comprising an acid or a solid acid, to produce a catalyst-oil mixture;
C) applying RF or microwave energy to at least one of the catalyst, the plant oil, the animal oil, the combination thereof, and the catalyst-oil mixture; and
D) cracking at least one of the plant oil, the animal oil and the combination thereof to produce hydrocarbons as biofuels, wherein less than 10% (wt/wt) of the plant oil, the animal oil or the combination thereof is converted to a hydrocarbon off-gas.
